(19) United States
(12) Patent Application Publication
(10) Pub. No.: US 2010/0098732 A1
(43) Pub. Date: Apr. 22, 2010

(54) METHODS, PROCESSES AND COMPOSITIONS COMPRISING THE PROTEIN CALMODULIN (CAM) FOR TREATMENT OF DAMAGED OR AGING SKIN, AND/OR HAIR LOSS

(76) Inventor: Dorr Felt Tippens, Seattle, WA (US)

Correspondence Address: Dorr Felt Tippens, 7003 17th Ave NW, Seattle, WA, 98117, US

(21) Appl. No.: 12/449,916

(22) PCT Filed: Mar. 21, 2007
(86) PCT No.: PCT/IB2007/002725

§ 371 Date: Sep. 2, 2009

(51) Int. Cl. A61K 8/02; A61K 38/00

(52) U.S. Cl. 424/401; 514/12

(57) ABSTRACT

The invention relates to compositions comprising the calcium binding protein, calmodulin (CaM), and the use of this protein additive in compositions suitable for topical application for the maintenance and development of healthy skin; for improving the aesthetic appearance of skin; for treating fine lines, and/or wrinkles, and/or age spots, and/or minor wounds and burns, and/or calloused skin—through its facilitation of natural healing processes initiated by disease, injury or aging; for enhancing hair growth; and as a cosmetic treatment process comprising application to the skin the compositions, preferably on a daily basis. The compositions may comprise, but not be limited to, UV blocking agents and/or penetration enhancers and/or one or more bio-active agents. CaM can be formulated into therapeutic compositions as lotions, creams, ointments, sprays, gels, skin coverings and wound dressings.

### FIELD OF THE INVENTION

This invention relates to topical compositions containing the protein calmodulin (CaM) that can be used for: 1) improving the appearance of skin affected by natural aging processes or over exposure to solar radiation through the repair and maintenance of the skin; 2) facilitating minor wound and/or burn healing; 3) promoting hair growth.

### BACKGROUND OF THE INVENTION

The aging process is ordinarily expressed in the skin through changes such as dryness, wrinkles, keratoses, age spots and pigmented skin lesions. This process can be accelerated by over-exposure to UV radiation or other harsh environmental factors such as chemical exposure. Skin atrophy is a natural result of aging, but may be caused by either intrinsic or extrinsic factors such as natural chronological aging, photo damage, burns or chemical damage, or by exposure to pollutant s or allergens such as cigarette smoke. The epidermis and dermis thin and the density of some structural and connective proteins, such as collagen and elastin, are reduced as one ages. With the loss of subcutaneous fat, reducing support for the epidermis, fine wrinkles and discoloration appear deepening with time.

Skin is composed of two layers. The top layer, epidermis, is approximately 0.1 mm thick. The lower dermis typically ranges from 1 to 4 mm thick and contains small blood vessels, collagen, elastin and fibroblasts. The dermis provides structural support and nutrients to the epidermis as it contains an elaborate network of elastin fibers responsible for maintaining the skin's elastic properties. This supporting dermis thins with age and sun exposure. Through the process of actinic elastosis, the elastin network becomes hyperplastic and disrupted. This is the principal cause of wrinkle formation, discoloration and laxity of the skin. These age-induced fine wrinkles (exacerbated by repeated prolonged exposure to solar radiation) are the direct result of deterioration of the underlying dermis layer—fewer fibroblasts, less collagen and elastin, and less circulatory support. However, as new fibroblasts, endothelial cells and keratinocytes form, the skin can repair itself. Unfortunately, the skin becomes less able to do so as it ages. Agents which can reverse this process and which can accelerate the growth and repair of skin are needed.

Fibronectin, a glycoprotein, is involved in many cellular processes including tissue repair, cell migration, wound repair, and cell adhesion. The extra-cellular matrix form of fibronectin is made by fibroblasts, chondrocytes, endothelial cells, macrophages and certain epithelial cells. Through its cell adhesion capabilities, it anchors cells to collagen or to proteoglycan substrates. Fibronectin has been implicated as a protein important in mediating the anti-wrinkle and skim-firming effects of various treatments [Chi et al., (2002)]. Therefore, agents and compositions that increase the amount of fibronectin in tissue are needed to maintain, promote and repair skin. Such agents and compositions would be useful to treat, rejuvenate and restore the condition of aged and damaged skin.

One common approach for improving the appearance of skin has been through the stimulation of proliferating new epidermal cells. Consequently, the skin takes on a younger appearance as these new epidermal cells do provide more structure and retain more moisture. Chemical peels are a common means to this end. Although this does offer some cosmetic improvement, it does not effect the more significant, supporting dermal layer.

Another prominent treatment of aged/photo damaged skin is through the topical treatment of tretinoin (Retin-a). It increases the epidermal turnover time stimulating the growth of those cells thereby reducing the appearance of fine wrinkles. However, it does not compensate for the equivalent reduction of melanocytes which help in the prevention of UV induced skin damage possibly increasing UV damage susceptibility. Individuals using tretinoin are cautioned to avoid unnecessary exposure to the sun. Additionally, it has been reported there is no observable change nor significant effect upon the dermis with this treatment [Weiss et al., 1988].

Cosmetic compositions that include alpha or beta hydroxy acid (AHA or BHA) as the active ingredient, are well-known in the art. Useful in improving skin tone, reducing fine lines, enhancing moisture retention and development of smooth skin, application of AHA/BHA results in more youthful looking skin through the accelerated replacement of skin cells. Unfortunately, both AHA and BHA act most effectively under acidic conditions. In order to improve skin condition, users must tolerate skin irritation caused by the acid present in the AHA/BHA products. This is contra-indicated for damaged and stressed skin and applying these products to sensitive areas of the body, such as the face, comes with marked caveats. Skin irritation is a major concern to manufacturers and users to these products.

Calcium is a significant mediator of a wide variety of intracellular physiological processes including gene regulation, DNA synthesis, cell cycle regulation, glycolysis for energy production in muscle contraction, as well as neurotransmitter release. These effects are initiated upon the release of stored calcium and its concomitant binding to calcium-binding proteins which further and specifically activate other proteins leading to physiological effects.

The present invention relies on calmodulin (CaM) protein, the most common effector of calcium effects, in topical compositions; thereby facilitating healing and regenerating of both epidermal and dermal layers as well as increasing blood flow in the dermal layer through its activating effect upon nitric oxide synthetase promoting hair growth.

Calcium exists as a gradient across the plasma membrane, with extracellular concentrations being about 10,000 times higher than intracellular ones. External signals can lead to transient increases in intracellular calcium concentrations. This ion gradient serves as a switch meditated by CaM and other calcium binding proteins in regulating many cellular processes (Eldik and Watterson, 1998; Jurado et al., 1999]. CaM undergoes a reversible conformational change upon its binding state to calcium, enabling it to bind to specific proteins for a specific response. Calcium serves as an intracellular signaling system by acting as a diffusible second messenger to the initial extra- and intracellular signals [Eldik and Watterson, 1998] and CaM is the most common mediator of calcium effects in eukaryotic cells [Rogers et al., 1996]. Increased calcium concentrations lead to calcium binding by CaM and its interaction upon regulatory proteins turn the calcium signal into a biological response.

Intracellular CaM concentrations are usually very high ranging from 2-5 μM in most cells to about 50 μM in brain [Elwess et al., 1997]. While the estimated total CaM concentration inside cells is ˜1-10 μM, free CaM concentration is significantly lower since it is associated with apo-CaM-binding proteins (Gerendasy, 1999; Jurado et al., 1999). The binding of calcium to CaM induces a conformational change in the protein that regulates its interaction with over 100 different proteins. Depending upon its calcium binding state, CaM serves as a switch upon these enzymes controlling a multitude of cellular processes including, but not limited to, gene regulation and DNA synthesis, cellular migration, cytoskeletal organization and intracellular movement and its effects on cell cycle progression and mitosis, signal transduction, ion homeostasis, exocytosis, metabolic regulation, inflammation, apoptosis, short-term and long-term memory, nerve growth, immune response and muscle contraction and glycolysis. The muscle contraction-relaxation cycle can be switched through CaM's calcium binding state by variations in the cytoplasmic calcium concentration ranging from 1 μM (inactive) to 100 μM (active).

The present invention is directed to treatments of a variety of conditions related to the skin: for sun damaged and/or aged skin; for minor abrasions and burns; and for hair loss—by targeting the cells of both the epidermal and the supporting dermal layers of skin. A composition containing available CaM would stimulate fibroblast proliferation and promote their production of collagen and elastin, thereby regenerating the underlying dermal layer. In addition, the CaM composition would stimulate the production of epidermal cells. Additionally, the invention's unique CaM composition mediates vascular relaxing effects of endothelial cells promoting hair growth where restricted vascular supply has interfered with normal growth. The effect of CaM would be synergistic with other skin protectants and stimulants commonly utilized in cosmetics and well know to the art.

Disclosed herein is a composition suitable for topical application to the skin, comprising, in a physiologically acceptable medium CaM with or without other components such as facilitators of CaM permeability, protectants, and/or other known stabilizers, regeneration additives, antibiotics and other bio-affecting agents.

### SUMMARY OF THE INVENTION

This invention relates to a novel method for the maintenance of healthy skin and the treatment and improvement of age-related and photo-damaged changes to the skin such as fine lines and wrinkles, keratoses, age spots and pigmented skin lesions. It is a further object of this invention to treat minor bums and wounds. As well, this invention covers a unique approach to promoting hair growth. These topical compositions, methods and processes include calmodulin (CaM) and may as well comprise a permeation enhancing amount of one or more penetration enhancers and one or more other bio-affecting agents which penetrate into the underlying tissues and into the vascular network of the skin. These unique CaM compositions, in various aspects of the invention, are useful in strengthening, firming, rejuvenating and restoring the condition of the skin to a more healthy and youthful appearance. Other tailored compositions containing CaM promote minor wound and wound healing. Still others can be used to promote hair growth, particularly on the scalp. CaM protein is a ubiquitous 2nd messenger that activates a wide variety of biochemical events. The inventor has a proprietary process for making CaM in large quantities affording the use of CaM in these compositions. Topical lotions, gels, ointments, spray and various cosmetics and dressings containing the CaM compositions are contemplated, as are methods of using the CaM compositions.

### DETAILED DESCRIPTION OF THE INVENTION

Calmodulin (CaM) is a ubiquitous, small, acidic, calcium-binding protein approximately 148 amino acids long (16,706 Dalton.) It typically binds a total of 0, 2, or 4 calcium ions at its four “EF-hand domains,” each of which can bind a calcium ion. Found in all eukaryotic cells, CaM can have different subcellular locations and is located within cellular and organelle membranes as well as in the plasma and saliva. CaM is the most important transducer of intracellular calcium signals through the second-messenger system. It binds and regulates different proteins through its stearic transformations due to its calcium-binding state. As early as 1981 calcium activated CaM interaction with a range of enzymes including myosin light chain kinase, fodrin, CaM kinase II, phosphodiesterase and inositol 1,4,5-trisphosphate kinase 1 was reported proving its strong role in many physiological processes of importance, such as glycolysis, muscle contraction, cell division, cell growth, exocytosis and endocytosis [Haiech et al., 1981].

CaM can undergo post-translational modifications, such as phosphorylation, acetylation, methylation and proteolytic cleavage, each of which can potentially modulate its actions. There are over a hundred proteins known to bind CaM where through this interaction it regulates the activity of a plethora of enzymes and non-enzyme proteins including the calcium pump, numerous specific protein kinases and phosphatases, cyclic nucleotide phosphodiesterases, spectrin, histones, and tubulin effecting many biochemical responses to calcium levels within cells. It thereby controls many cellular processes, metabolic and otherwise, including protein phosphorylation and dephosphorylation, cyclic-nucleotide formation and breakdown, cytoskeletal rearrangement and intracellular motility, gene transcription, inflammation, apoptosis, muscle contraction, both short-term and long-term memory, nerve growth, immune response and membrane potential. Certain studies suggest that CaM participates in the reparation of deoxyribonucleic acid and that would be involved in the reversal of aging or photo-damaged skin.

CaM is highly conserved across all eukaryotes and its expression is essential for biological cells to progress through mitosis. The inventor has played a significant role in the discovery of some of CaM interactions beginning his research with CaM in the 1970's in the laboratories of Nobel Laureate, professor Edmund H. Fischer. Later in the decade, and through the early 1980's, the inventor through his company, Enzymatics Inc., through a proprietary process, made CaM available to the research community. Enzymatics Inc. has been the sole supplier of CaM to BioRad Laboratories, a large international distributor of products to the research community.

CaM concentration varies throughout the body. Intracellular concentration within the brain or testes is about 20-25 nM and about 10 nM in the heart and taste bud membranes [Law et al., 1985]. Whereas in the saliva CaM concentration in normal subjects is about 4.5nM which is about 10 times higher than that found in serum although it has been reported in the literature that intracellular available CaM concentration of ˜10 uM under resting conditions in a human kidney cell line [Black et al., 2004]. Efficient activation of low-affinity targets (>/=100nM) occurs only where free calcium activated CaM concentrations can be locally enhanced [Persechini et al. 1999].

The primary embodiment of the present invention are compositions that comprise CaM, as well as any CaM which has undergone any post-translational modifications such as, but not limited to, phosphorylation, acetylation, methylation and proteolytic cleavage, each of which can potentially modulate its actions. The method and process of the use of CaM in these compositions are also significant objects of this invention.

The compositions of the invention are preferably applied to the skin or the mucous membranes.

The compositions of the invention can be cosmetic or pharmaceutical compositions.

The CaM compositions can be introduced into a composition that is intended for the moisturizing and/or making up of the skin.

CaM containing compositions, the core embodiment of the invention, can be used as an agent that regulates the impairments of the skin including the treatment of dry skin, hyperkeratosis, parakeratosis, psoriasis, ichthyoses, and neoplasias.

Another embodiment of the invention is the use of some CaM compositions, methods, and processes to prevent, treat and fight against the signs of aging skin and/or the effects of A or B type UV radiation.

Another embodiment of the invention are CaM compositions, methods, and processes intended to facilitate hair growth where normal hair growth has been impaired through poor vascularization. The primary target for this embodiment of the invention would be for the scalp.

An unusual aspect of the protein CaM is its ability to withstand 6 M urea. Typical concentrations of urea containing topicals are about 25% urea (3.6 M); less than ½ of the concentration which CaM readily renatures from. Urea is a common component in topical compositions for the treatment of dry, cracked and calloused feet. Urea can solubilize the otherwise highly resistant keratin proteins which are the principal constituent of these thickened areas of the skin. CaM compositions as covered by this invention with concentrations of urea capable of dissolving the thickened keratins, still allow the penetration of a stable and active CaM to the underlying skin layers. Urea-containing, CaM compositions tailored for the care and treatment of the feet is, therefore, another significant application of this invention

CaM used in this invention may be derived from any source, including, without limitation, from recombinant and non-recombinant sources, plant or animal, over a wide range of concentrations inclusive of normal ranges and those extending above and below normal bio-active concentrations. This inclusive, wide range of CaM concentrations under consideration is due to the effective concentration of CaM being a function of what the functional target addressed is; the vehicle in which it is dispersed; how volatile this carrier is (evaporation of the carrier increases the relative concentration of CaM); how much of the composition is applied to any given area; and how effective the permeation enhancers, if used, are. A CaM concentration in the range of 0.1 to 100 n M would have the most bio-affective properties and would be the most likely concentration range used in the various formulas. However, other concentration ranges of CaM may be used in the compositions and are being considered by the inventor with the possibility of factors of 1,000 fold, greater or smaller, applied in certain compositions due to, for example, various permeability factors, specific target(s), type of carrier—among other considerations. Other bio-affecting agents, and their concentrations, in the compositions can also vary greatly and they too will be dependent upon may factors, e.g. type, bioavailability, efficacy, potency, Kd (binding strength), application surface area, which composition is used and the amount and type, if used, of penetrating agent(s) used. The expected concentration of the various bio-affecting agents under consideration will vary from about 0.1% to 40% of the total composition, either suspended or dissolved.

Further disclosed in the detailed description of this invention are examples of other pharmaceutically acceptable ingredients, in addition to CaM, that may be a part of specific compositions tailored for specific purposes and serve by example, not by way of limitation, of ingredients that are compatible with the skin, the mucous membranes, the nails and the hair. And also disclosed are the methods and processes uniquely described through the use of CaM in these compositions. CaM will be mixed with pharmaceutically acceptable carriers or excipient(s) at wide ranges of doses, detailed above, to treat or ameliorate a variety of skin disorders, including hair loss, as well as to maintain healthy looking skin. Such compositions may optionally contain diluents, fillers, salts, buffers, stabilizers, solubilizers, and other materials well known in the art. The term “pharmaceutically acceptable” means a non-toxic material that does not interfere with the effectiveness of the biological activity of CaM. The characteristics of the carrier will depend on the route of administration and which physiological target(s) upon which CaM acts is being selected.

The composition may further contain other agents which either enhance the activity of CaM or complement its activity or use, including calcium. Such additional factors and/or agents may be included in the compositions to produce synergistic effects with CaM and/or aid in selecting its target(s).

The compositions will be blended into skin compatible vehicles such as hydrophobic and hydrophilic lotions, ointments, creams, sprays or gel-based vehicles. Such pharmaceutically acceptable vehicles are well known in the art and commercially available for formulation of active ingredients into a suitable form for topical application.

### Wound Healing

Calcium has an established role in the normal homeostasis of mammalian skin and serves as a modulator in keratinocyte proliferation and differentiation. Gradients of calcium concentration from 0.5 mM in the basal layer to >1.4 mM in the stratum granulosum are consistent with migration patterns in response to minor abrasion (normal wear). Dermal fibroblasts require calcium but are approximately 100 times less sensitive than keratinocytes. In wound repair, calcium predominantly effects Factor IV in the hemostatic phase, but is required in epidermal cell migration and regeneration patterns in later stages of healing [Lansdown et al., September-October 2002]. Metalloenzymes have an important role in repair and regenerative processes in skin wounds depending on the phase in the healing cycle. Calcium levels peak about 5 days into wound healing associated with inflammation, granulation and epidermal cell proliferation. CaM levels are highest in maturing keratinocytes and in sebaceous gland cells of normal skin. However, it is in greater concentrations in the epidermis near wound margins and in re-epithelializing areas in concert with the highest calcium ion levels [Lansdown et 1999]. Providing added CaM through these compositions would insure adequate amounts of CaM for these wound healing processes and make it available immediately.

Wound healing requires fibroblast migration which synthesize and organize new extracellular matrix proteins. This process is dependant upon myosin ATPase activation which develops cytoplasmic actin-myosin contraction. Myosin ATPase activity is optimized through phosphorylation of myosin light chain. CaM-dependent myosin light chain kinase (CaM-MLCK) is a key enzyme involved in this phosphorylation. The phosphorylation of myosin light chain by CaM-MLCK and subsequent myosin ATPase activation affects granulation tissue fibroblast behavior and contributes to wound contraction. Myosin ATPase activity also generates actin-myosin contraction within fibroblasts and is involved in ATP-induced cell contraction, the generation of focal adhesions, fibroblast migration, fibroblast populated collagen lattice (FPCL) contraction, and wound contraction. It was reported that CaM inhibitors fluphenazine and the naphthalenesulfonic acid derivative W7 blocked rat open wound contraction and fluphenazine also delayed re-epithelialization at the wound site. These findings support the idea that fibroblast CaM-MLCK activity is essential for tissue repair [Levinson et al., October 2004]. Another study using transformed cell lines showed CaM like protein (CLP) displayed a striking increase in both number and length of filopodia and increased motility in a wound-healing assay helping in terminal differentiation as cells migrate toward the upper layers and establish new adhesive contacts [Bennet et al., 2006]. One control of cellular migration is through the regulation of the contraction of the trailing edge of migrating fibroblasts. CaM and MLCK, one of the many enzymes it acts upon, are involved leading to phosphorylation of the myosin light chain at the trailing end. This trailing edge contraction is critically regulated by Ca2+ and its subsequent activation for the control of fibroblast cell migration [Yang et al., 2005]. Comparing caldesmon (CaD) regulation by CaM a study was performed using a mutant form of CaD which could not bind to CaM but had little effect upon CaD binding to actin filaments nor its ability to inhibit actin/tropomyosin activated heavy meromyosin ATPase. Stable expression of mutant CaD expressing cells disrupted assembly of both stress fibers and focal adhesions, altered cell morphology and slowed cell cycle progression. Critically, these mutant expressing CaD cells exhibited motility defects in a wound-healing assay adding to the preponderance of evidence and results of the critical role CaM plays in wound healing [Li et al., 2004]. Two immediate early genes (IEGs) are expressed in endothelial cells at a wound edge. The transcriptional activation of the IEGs depend, in part, upon CaM-dependent protein kinase. Blocking this activation produces a long-term reduction of cellular motility [Tran et al., 1999]. By supplying the wound area with calcium activated CaM, the CaM-MLCK would be activated, favoring wound healing.

Many other studies have shown the critical role CaM plays in wound healing. During wound healing, a cellular-collagen complex is formed called the fibroblast-populated collagen lattice (FPCL). As the wound further heals, the FPCL reduces in size for wound closure. This is called lattice contraction. Elevated levels of myosin ATPase are required for maximal lattice contraction. This activity is increased when the myosin light chains are phosphorylated by myosin light chain kinase (MLCK). MLCK is activated by CaM [Ehrlich et al., 1991]. Failure of corneal epithelial wound closure was found to be correlated with a general decrease in CaM induced by certain anesthetics [Higbee et al., 1989]. A significant augmentation of CaM was found in granulation tissue development during the early stages of the wound healing process [Mizumoto, 1987]. During oral mucosal regeneration CaM dependent cAMP phosphodiesterase was significantly elevated at wound sites [Fine et al., 1986]. Cellular migration and cellular adhesion was found to be blocked by CaM inhibition upon cultured rat corneal epithelial cells [Soong et al., 1985]. CaM agonists inhibited proliferation of keratinocytes and fibroblasts [Al-Ani et al., 1988]. Using fluorescent analogues, it has been found that CaM is activated during serum activation of quiescent fibroblasts as well as during the transverse fiber contraction during wound healing. In the same study, migrating fibroblasts in the later stages of wound healing had a correlating activation of CaM [Hahn et al., 1992].

In summary, CaM's role in wound healing has been well documented over the years. The use of extraneous CaM in the CaM containing compositions covered by this invention and through the methods and procedures outlined therein, would have significant impact upon wound healing.

### Burn Healing

CaM has been found to have a significant role in the mitogenicity of burn blister fluid in cultured keratinocytes and fibroblasts. Blister fluid CaM levels are three times greater than in serum. Corresponding concentrations of both epidermal growth factor and platelet-derived growth factor, however, are significantly lower. These in vitro studies suggest burn blister fluid promotes wound healing. The researchers concluded locally released CaM contributes to this effect suggesting it would be beneficial to leave burn blisters intact to preserve this higher level of CaM [Wilson et al. 1994]. An important embodiment of this invention is to supply CaM compositions to skin burns promoting this beneficial effect. Such compositions would be significant with burns where the burn blisters had ruptured or where no blister formations occurred. In effect, the CaM containing compositions covered by this invention would serve as a surrogate, CaM-containing, blister fluid in the treatment of skin burns.

### Hair Growth Stimulation

In addition to the role CaM plays in rejuvenating skin and treating minor wounds and burns, it also, specifically, has a role in promoting hair growth. U.S. Pat. No. 5,468,476 [Ahluwalla et al., 1995] is based on this phenomenon through the use of a composition to reduce unwanted hair growth through the topical application of an inhibitor of nitric oxide synthetase (NOS) to the skin. The present invention works on the opposite pathway. Rather than reducing the presence of unwanted hair by inhibiting NOS, it provides CaM, an activator of NOS, to encourage healthy hair growth. Nitric oxide (NO) mediates vascular relaxing effects of endothelial cells. The enzyme NOS, which forms NO in the body, requires CaM for activity [Bredt et al., 1990]. Other U.S. patents such as U.S. Pat. No. 5,869,539 [Garfield et al., February 1999] used compositions containing carriers for NO rather than using the novel concept of applying CaM as the direct activator of nitric oxide synthetase. In addition to stimulating hair growth, NO improves wound healing and skin texture which relates to the other embodiments of this invention's CaM containing compositions.

The components of the method and composition of the present invention will be discussed separately due to the importance of the unique and novel use of CaM in topical compositions and illustrate its synergistic application through the various methods and compositions described for the maintenance of healthy skin, prevention and treatment of aging or damaged skin as well as its novel use in compositions for the treatment of hair loss. The topical composition of the present invention may comprise one or more penetration enhancers and/or one or more bio-affective agents.

### Penetration Enhancers

The present invention may use a penetration enhancer or permeation enhancer in its composition to increase the permeability of the skin to CaM allowing its penetration to the tissues and bloodstream and/or its availability intracellularly. A chemical skin penetration enhancer increases skin permeability by reversibly altering the skin's physiochemical nature thereby reducing its diffusional resistance. Physical penetration enhancers disrupt the skin surface allowing faster penetration. Minor wounds and abrasions serve this purpose by their very nature.

Chemical Penetration Enhancers

There is now a growing number of OTC and prescription medications available via skin patches to treat a growing list of diseases and conditions through the incorporation of safe penetration enhancers allowing macromolecules to diffuse through the skin. Birth control, weight loss, pain relief, hormone replacement therapy, heart disease, Parkinson's disease, nicotine replacement, sexual desire, dental pain, shingles, and osteoporosis are examples of conditions now being treated through the delivery of medications through transdermal skin patches. Patches are now being developed for the treatment of asthma, cancer, depression, diabetes, hyperactivity, auto-immune dysfunction and migraine. The technology is very prevalent in the skin care industry where therapeutic, cosmetic and even monitoring skin care patches are widely accepted.

The outermost layer of the skin, the epidermis, approximately 100-150 μm thick, is constantly replenished and has no blood flow. It includes a layer within it known as the stratum corneum. This layer is what transdermal delivery needs to bypass as its composition serves as a two-way penetration barrier keeping fluid components within the body and foreign substances out. Below this layer is the dermis, 1-4 mm thick, containing nerves, blood and lymph vessels, sweat and sebaceous glands and hair follicles. By penetrating the stratum corneum and cellular membranes, macromolecules, including polypeptides the size of CaM, can enter the blood stream and/or cells. Hair follicles on the skin can act as shunts to bring the composition from the skin's surface down into the dermis. Efficient transdermal drug delivery of large hydrophilic drugs has been shown to be greatly facilitated by a short synthetic peptide, ACSSSPSKHCG, through intact skin. In the study, insulin, co-administered with the peptide resulted in elevated systemic levels of functional insulin for at least 11 hours. Similar results were achieved with human growth hormone. The transdermal-enhancing activity of the peptide was sequence specific and dose dependent with no interaction with insulin enabling penetration of insulin deeply into hair follicles. This peptide creates a transient opening in the skin barrier to enable macromolecular drugs to reach systemic circulation [Chen et al., 2006]. This new peptide penetration enhancer, along with other peptides yet to be discovered with similar effects, offers reduced side effects and chemical sensitivity as compared to other types of chemical permeators and is being considered as an integral component in the novel, topical compositions containing CaM covered by this invention. Below the dermis is a layer of connective tissue and fat cells that form the subcutaneous layer.

Several safe, non-aqueous solvents increase penetration. Hundreds of different chemical compounds have been cited as skin penetration enhancers. Most of the compounds are generally recognized as safe (GRAS) ingredients that would often be considered inert by a formulator [Osborne et al., 1997]. Examples of chemical penetration enhancers include: alcohols, ethanol and isopropanol most commonly incorporated; polyols, several including glycerols and glycols; sulfoxides, such as dimethylsulfoxide (DMSO); esters, not limited to isopropyl myristate/palmitate, ethyl acetate, butyl acetate, methylproprionate, and capric/caprylic triglycerides; ketones; amides; oleates, and various surfactants and detergents both ionic and non-ionic, such as sodium lauryl sulfate; various alkanoic acids, such as caprylic acid; lactam compounds; alkanols; dialkylamino acetates, and admixtures thereof.

Recently, more research has been applied to the transdermal delivery of macromolecules of the approximate size of CaM by up to ˜100 fold without inducing skin irritation, a critical criterion for cosmetics and sensitive skin. Examples of macromolecules tested were heparin, leutinizing hormone and oligonucleotides. One of these mixtures was the common detergent sodium lauryl sulfate in conjunction with phenyl piperazine and their use for delivering macromolecules from a transdermal patch was found efficacious [Karande et al., 2004].

A number of patents disclose the use of penetration enhancers to deliver medications transdermally: Grasela et al., U.S. Pat. No. 5,837,289; Catz et al., U.S. Pat. No. 5,238,933; Sharma et al., U.S. Pat. No. 5,229,130; Tsuk, U.S. Pat. No. 4,933,184; Havemeyer et al., U.S. Pat. No. 4,342,784; Rajadhyaksha, U.S. Pat. No. 5,482,965; Samour, U.S. Pat. Nos. 5,620,980, 5,807,957; and Petrus et al., U.S. Pat. No. 6,573,299.

Physical Transdermal Delivery Enhancers

Low-frequency ultrasound increases skin permeability (sonophoresis) to many compounds and/or drugs, including high molecular weight proteins, by several orders of magnitude. Therapeutic doses of proteins such as insulin, interferon gamma, and erythropoeitin have been delivered using low-frequency (20 KHz) ultrasound as a noninvasive substitute for traditional methods of drug delivery, such as injections [Mitragotri et al., 1995]. Many ultrasound devices are available commercially which can be used in the method described herein. Ultrasonic devices used by dentists to clean teeth have a frequency between about 25 and 40 KHz. Commercially available, portable, ultrasound tooth-brushes make use of a small sonicator contained within the tooth-brush (Royal Philips Electronics). This technology allows effective transdermal transmission by means of skin systems not larger than a wristwatch. Using this, and similar techniques, application to the skin of CaM, alone or in combinations with a carrier(s); penetration enhancer(s); lubricant(s); bio-affecting components; and/or other pharmaceutically acceptable agents can deliver CaM to underlying target cells.

The use of electric potential to move charged therapeutic molecules across the skin, electrotransport, has been well developed over the past decade. Since the permeation rate of drugs in electrotransport systems is proportional to the applied current, the dose of the delivered drug can be easily manipulated by controlling the electrical current. The technology enables the delivery of relatively high-molecular weight entities, including peptides and proteins, such as CaM.

Other transdermal innovations include macroflux technologies utilizing microprojection patches. The main component of the microprojection patch is a metallic disk, typically titanium, with an adhesive back. The disk consists of an array of microprojections that are coated with medicinal substances. There are often 300 microprojections per cm of disk surface. These penetrate just the 10 μm to 25 μm-thin layer of dead cells of the stratum corneum creating microchannels—large enough to permit the transport of large molecules to the physiologically active deeper layers of the epidermis. This viable epidermis has no capillaries, however the underlying, extensive vascular network of the dermis is immediately adjacent and takes up the medicinal substances rapidly. The microprojections are too small to cause pain. This technology offers a needle-free and painless transdermal drug delivery of large-molecular-weight compounds such as CaM.

Although penetration enhancers, either physical or chemical, facilitate penetration through the skin and absorption into the vascular network of the underlying dermis, abrasions, for example, may already allow permeation of CaM and, as well, tend to be more sensitive to some of the chemical permeation enhancers.

This topical administration offers a significant advantage over oral administration of therapeutic agents by overcoming the difficulty of poor gastrointestinal absorption and/or breakdown due to digestion, by using a lower dosage than required orally, and allowing more of the active agents to provide therapeutic relief at the targeted sites.

### Bio-Affecting Agents

The present invention relates to novel compositions for topical application and delivery of bio-affecting agents through the protective outer layer of the skin, into the underlying tissues and into the vascular network of the targeted body part through the inclusion of CaM as part of the composition. The term “bio-affecting agent” refers to any chemical substance or formulation which beneficially affects the body. In addition to CaM, the compositions may include components of bio-affecting agents. These may comprise any combination of, however not be limited to but rather to serve by example:

Lutein and zeaxanthin, a form which can be converted from lutein, and/or other carotenoids protect the skin by absorbing UV light, block the activity of peroxide radicals, and inhibit LDL oxidation thereby protecting cell membranes from this and other free radical damage—the use of carotenoids, such as these, is well known in the industry [Baranowitz et al., U.S. Pat. Nos. 5,310,764 and 5,457,135; Bernstein et al., U.S. Pat. No. 5,873,831; and Bernhard et al., U.S. Pat. No. 5,780,693]—none use the method and/or composition of the present invention.

Nitric oxide synthase inhibitors (NOSI) including resveratrol: NOS is inducible by endotoxins, cytokines, growth factors (often associated with keratoses and carcinomas) and immune complexes. Its over expression can result from increased levels of tumor necrosis factors and inflammation inducing cytokines. Nitric oxide plays an important role in autoimmunity and inflammation. NSAIDs, zinc compounds and tetracycline are among the more common chemicals which inhibit expression of the protein, NOS. These are but a few of the ingredients commonly used by the industry being considered for use in the compositions with the novel added composite, CaM. Resveratrol, an antioxidant found in wine, in addition to inhibiting NOS, can also inhibit the action of cyclo-oxygenase (COX). It shares this inhibitory function along with non-specific NSAIDs. COX 1 is involved with epithelial turnover and COX 2 with prostaglandin synthesis which is a mediator in pain perception, inflammation, fever, platelet aggregation and smooth muscle contraction. Resveratrol also is used to treat peripheral vascular diseases and peripheral diabetic neuropathy [Sato et al., 1997; Tsai et al., 1999; Kawada et al., 1998]. However, as an example of the different use and effects of the compositions covered in this invention, NOS inhibitors would be used as a component in some compositions and in others, not. For the case of hair growth enhancement compositions, methods and/or procedures, the specific inhibition of NOS through the addition of NOS inhibitors would not be incorporated as, in that case, the effect of NOS activation by CaM, and therefore not inhibition by other composites, is the primary goal and one of the embodiments of this invention.

Alpha and beta hydroxy acid (AHA/BHA) serve as exfoliates smoothing skin and assisting in cell turnover. AHA/BHAs have been widely applied by the skin care and cosmetic industries. They also can act as permeation enhancers. AHAs work partly by sloughing off dead cells on the skin surface and are best for sun damage or a thickened epidermis. AHAs also thicken the dermis and increase hyaluronic acid which helps in the adhesion of cells. In this way, AHA/BHAs are effective in smoothing out fine lines and wrinkles of the skin. AHAs are also effective in treating most age spots and dry skin. Typical AHA concentrations range between 5% to 10%. They are most efficacious in acidic compositions with a pH of 3-4, a range compatible with CaM. BHAs can cut through the fatty layer of skin assisting in exfoliation and clearing skin pores. Typical BHA concentrations are between is 1% to 2%. They too are most effective in an acidic environment with a pH range of 3-4. It is this acidic pH, although a stable range for CaM, that is its most serious caveat for the incorporation of AHA/BHA in topical compositions. AHA/BHA may be present as free acids, or as peroxides, lactones, amides, esters or salts of organic or inorganic bases. CaM works synergistically with AHA and/or BHA, but for those with sensitive skin, a composition containing CaM in the absence of AHA/BHA would be recommended and is one of the advantages of this invention. A number of patents disclose the use of exfoliates and other ingredients to treat aging skin [Parah et al., U.S. Pat. No. 5,254,343: Yu et al., U.S. Pat. Nos. 5,886,042 and 5,889,054; Scancarella et al., U.S. Pat. No. 5,643,587; Duffey et al., U.S. Pat. No. 5,676,956; Cohen et al., U.S. Pat. No. 5,876,736 and Petrus et al., U.S. Pat. No. 6,573,299]. None of the above cited patents teach or suggest the use of the method or composition outlined in the present invention including CaM in the composition.

### Anti-Inflammatory Agents

Inflammation accelerates the aging process and is believed responsible for many of the changes that occur in aging and/or photo damaged skin. An acute inflammatory reaction occurs after cellular injury from either bums, UV radiation, and/or trauma caused by minor wounds or chemicals. One of the first reactions to the skin and underlying tissue is a change in both the microvasculature and the interstitial areas, those fluid filled areas between cells and tissues. Following vasodilation there is an induced permeability of the blood vessels causing fluid to pass through into the associated tissues causing edema. Finally, white blood cells infiltrate into this swollen tissue. However, due to this increased blood flow and permeability, the inflamed skin becomes more amenable to penetration enhancers delivering the bio-affective agents. Histamines facilitate vascular changes along with prostaglandins which also induces platelet aggregation. There are other chemical mediators including leukotrienes, serotonin and lymphokines. Some anti-inflammatory bio-affective agents coupled with CaM that may be included in the present invention include the following as examples of those being considered:

N-acetylcysteine and other amino acid derivatives of cysteine have antioxidant properties with anti-inflammatory effects. They interact with peroxides and leukotrienes reducing free radicals and thereby reducing inflammation and the promotion of healing. In combination with glutathione and ascorbic acid, synergistic results have been found. Their incorporation has been well known in the art [Bissett et al., U.S. Pat. No. 5,821,237; Epstein, U.S. Pat. No. 5,306,731; Fleming et al., U.S. Pat. No. 5,871,769; Mason et al., U.S. Pat. No. 5,691,380; Morgan, U.S. Pat. No. 4,708,965; Noble et al., U.S. Pat. No. 5,766,873 and Repine et al., U.S. Pat. No. 5,596,011]. None of these cited patents use or suggest the novel inclusion of CaM in their compositions, which would synergistically complement the effects of these anti-inflammatory, bio-affective agents.

Ascorbyl Palmitate, a synthetic ester of vitamin C (ascorbic acid), is fat soluble, neutral in pH, and non-irritating to the skin. Hydrophobic, it penetrates the skin barrier more readily than ascorbic acid. It is effective both as a pre and post treatment to UV photo-damaged skin. Vitamin C is the chief antioxidant in skin and can be significantly depleted after relatively short exposures to UV. Topical ascorbyl palmitate has been found to be a powerful anti-inflammatory agent within the cell, and stimulates collagen production and fibroblasts which help strengthen the dermis. Located along with vitamin E in the cell membranes, it can regenerate the vitamin E radical as opposed to ascorbic acid which lacks this hydrophobic advantage. The use of ascorbic acid and its derivative, ascorbyl palmitate, are well known in the art [Fallick, U.S. Pat. No. 5,846,996 & 5,945,447; Ito et al., U.S. Pat. No. 5,937,790; Leiner, U.S. Pat. No. 5,470,874; Mausner, U.S. Pat. No. 5,922,331; Meissner, U.S. Pat. No. 4,772,591; Murad, U.S. Pat. No. 5,804,594; Perricone, U.S. Pat. No. 5,409,693 and Schinitsky et al., U.S. Pat. No. 4,938,969]. None suggest the use of CaM within their compositions or treatment.

Alpha-lipoic acid (ALA) is another potent antioxidant and anti-inflammatory agent that that has the advantage of being both hydrophobic and hydrophilic: a universal antioxidant. A 1% lotion can decrease fine wrinkles, increase levels of the free-radical scrubbers and anti-inflammatory agents vitamins C and E, as well as serve as a neuroprotective agent. Well known in the art [Blaschke et al., U.S. Pat. No. 5,281,722; Conrad et al., U.S. Pat. No. 5,650,429; Kalden et al., U.S. Pat. No. 5,334,612; Koltringer, U.S. Pat. Nos. 5,118,505, 5,532,269; Urich et al., U.S. Pat. No. 5,728,735 and Weischer et al., U.S. Pat. No. 5,569,670]. None suggest the novel use of CaM neither by method nor compositions as described in the present invention.

Glutathione is a tri-peptide made up of glycine, glutamic acid and cysteine. Its use as an antioxidant and free-radical scrubber has been well described for its effect of regulating skin wrinkles and for increasing the metabolic rate of aged or damaged cells [Ames et al., U.S. Pat. No. 5,916,912; Blank et al., U.S. Pat. No. 5,789,396; Fleming et al., U.S. Pat. No. 5,939,394; Hersh, U.S. Pat. Nos. 5,667,791 & 5,922,346; Ohlenschlager et al., U.S. Pat. No. 5,925,620; Riley et al., U.S. Pat. No. 5,925,348]. Again, none suggest the novel use of CaM within their compositions.

Methyl-sulfonyl-methane (MSM) is a dimethyl sulfone (DMSO) derivative with an extra oxygen molecule lacking the lipid-solubility of DMSO. Both are well known and effective anti-inflammatory agents. MSM can be coupled with another penetration enhancer. MSM gives up its sulfur to form the amino acids methionine and cysteine which are rich in the dermal connective tissue. Penetrating the skin's surface, MSM's anti-inflammatory effects complement its use to moisturize, soften and rejuvenate dry, aging or damaged skin [Herschler et al., U.S. Pat. Nos. 4,296,130, 4,477,469, 4,863,748, 4,973,605 and 5,071,878]. None of his patents suggest the use of CaM in those compositions.

Zinc (Zn) compounds have anti-inflammatory and anti-infective properties [Petrus et al., 1998]. Zn is an essential trace element for humans and is required for many physiological processes. Among many, these include skin integrity, wound healing, growth and immunity. Several hundred enzymes have been described which require Zn. Zn has an inhibitory effect on histamine release from mast cells. Zn compounds are acknowledged as anti-inflammatory agents, as astringents, are beneficial in wound healing, and have antimicrobial, antifungal and antiviral activity. Zn is the active agent in formulations to treat skin disorders, decubitus ulcers, abrasions, and has a tightening effect on sagging or loose skin. Zn stabilizes the cell membranes and inhibits the formation of free radicals. Zn also strengthens the integrity of blood vessel walls by reducing the membrane permeability and in helping stop bleeding. Many patents containing Zn in their compositions are reflected in the art [Bryce-Smith, U.S. Pat. No. 5,792,449; Godfrey, U.S. Pat. No. 5,897,891; La Haye et al., U.S. Pat. No. 5,156,852; Turner, U.S. Pat. No. 3,856,941]. None suggest the synergistic nor novel incorporation of CaM in their compositions.

Aloe vera extracts, long used by our Native American population, has been well reported to have anti-inflammatory properties. It also acts as an anti-oxidant and has antiseptic properties. The present invention may include aloe vera in some of its compositions with CaM. Topical treatments with aloe vera were found to increase the synthesis of glycosaminoglycans and facilitate would healing and collagen synthesis [Chithra et al., 1998]. Heggers et al. (1997) found aloe vera improved wound healing and prevented dermal ischemia. Several patents illustrate the wide use of aloe vera in topical applications, however none discuss the inclusion or advantages of using it with CaM [Carpenter et al., U.S. Pat. No. 5,786,342 and Strickland et al., U.S. Pat. No. 5,824,659].

Other active components may be added to the composition to achieve the desired therapeutic effects to skin. Some of the active components may include, but are not limited to, a selection from a wide variety of therapeutic agents, known to provide beneficial effects when absorbed into the tissues and vascular network: vitamins, bioflavenoids, pyruvate, amino acids, dimethylaminoethanol, ubiquinones, minerals, lycopene, taurine, rutin, malic acid, skin whiteners and lecithin in addition to other additives such as water, alcohols, oils, glycols, preservatives, antioxidants, stabilizers, surfactants, emollients, anti-infective agents, adjuvants, thickening and gelling agents, anthocyanidins, proanthocyanidins, amino sugars, glycosaminoglycans, colorants, gums, esters, hormones, silicones, polymers, fragrances, sunscreens, salts buffers, polyols, other proteins and their derivatives, herbal extracts, detergents, waxes, amines, sugars, histidine, and the like and mixtures thereof along with other possible active agents where they might complement the integral and novel aspect of having CaM in these compositions in the form of a solution, suspension, cream, ointment, gel, film, spray or be contained within a dermal patch or the like.

Although embodiments of the invention have been described, a wide range of modifications are contemplated in the foregoing disclosure and in some instances, some features of the present invention may be employed without a corresponding use of the other features. Accordingly, it is appropriate that the appended claims be construed broadly and in a manner consistent with the scope of the invention. The over-riding scope of the invention and where it significantly differs from the current art is the incorporation of CaM into these compositions.

### REFERENCES

Al-Ani, A. M., Messenger, A. G., Lawry, J., Bleehen, S. S., MacNeil, S “Calcium/calmodulin regulation of the proliferation of human epidermal keratinocytes, dermal fibroblasts and mouse B 16 melanoma cells in culture,” British Journal of Dermatology 119(3):295 (September 1988).
Babu, Y. S., Sack, J. S., Greenhough, T. J., Bugg, C. E., Means, A. R. and Cook, W. J. “Three-dimensional structure of calmodulin,” Nature, 315:37-. (1985).
Barry, “Mode of Action of Penetration Enhancers in Human Skin,” J Controlled Rel. 6:85-97 (1987).
Bennett, R. D., Mauer, A. S., Strehler, E. E. “Calmodulin-like protein increases filopodia-dependent cell motility via upregulation of myosin-10,” J. Biol. Chem. (Nov. 26 2006); [Epub ahead of print].
Black, D. J., Tran, Q. K., Persechini, A. “Monitoring the total available calmodulin concentration in intact cells over the physiological range in free Ca2+,” Cell Calcium 35(5):415-25 (May 2004).
Bredt, D. S., Snyder, S. H. “Isolation of nitric oxide synthetase, a calmodulin-requiring enzyme,” PNAS 87(2):682-685 (January 1990).
Chen, Y., Shen, Y., Guo, X., Zhang, C., Yang, W., Ma, M., Liu, S., Zhang, M. & Wen, L.-P. “Transdermal protein delivery by a coadministered peptide identified via phage display,” Nature Biotechnology 24:455-460 (2006).
Chi, Y. S., et al., “Effects of the Chestnut Inner Shell Extract on the Expression of Adhesion Molecules, Fibronectin and Vitronectin, of Skin Fibroblasts in Culture,” Archives of Pharmacal Research, 25(4):469-474 (2002).
Chithra P., Sajithlal, G. B., Chandrakasan, G. “Influence of Aloe vera on the glycosaminoglycans in the matrix of healing dermal wounds in rats,” J Ethnopharmacol; 59(3):179-86 (January 1998).
Chithra P., Sajithlal, G. B., Chandrakasan, G. “Influence of Aloe vera on collagen turnover in healing of dermal wounds in rats,” Indian J Exp Biol 36(9):896-901 (September 1998).
Cleary, Gary W., “Transdermal Controlled Release Systems,” Medical Applications of Controlled Release (Langer and Wise, Editors, CRC Press 203-251, 1984).
Clegg and Vaz, “Translational diffusion of proteins and lipids in artificial lipid bilayer membranes. A comparison of experiment with theory,” Progress in Protein-Lipid Interactions Watts, ed. (Elsvier, N.Y.) Chapter 5:173-229 (1985).
Ehrlich, H. P., Rockwell, W. B., Cornwell, T. L., Rajaratnam, J. B. “Demonstration of a direct role for myosin light chain kinase in fibroblast-populated collagen lattice contraction,” J Cell Physiol 146(1):1-7 (January 1991).
Eldik L. V. and Watterson, D. M. “Calmodulin and Signal Transduction,” Academic Press, San Diego, Calif. (1998).
Elias, “The Microscopic Structure of the Epidermis and Its Derivatives,” Percutaneous Absorption: Mechanisms-Methodology-Drag Delivery (Bronaugh, R. L., Maibach, H., Editors, Marcel Dekker, New York,) 1-12 (1989).
Elwess, N. L., Filotoeo, A. G., Enyedi, A. and Penniston, J. T. “Plasma Membrane Ca2+ Pump Isoforms 2a and 2b Are Unusually Responsive to Calmodulin and Ca2+,” J Biol Chem 272(29):17981-86 (July 1997).
Eppstein, D. A. et al., “Alternative Delivery Systems for Peptides and Proteins as Drugs,” 5 CRC Reviews in Therapeutic Drug Carrier Systems 99 125 (1988).
Eppstein, D. A., “Medical Utility of Interferons: Approaches to Increasing Therapeutic Efficacy,” 7 Pharmacy International 195-198 (1986).
Finch & Schneider, eds. “Handbook of The Biology of Aging,” 1985, Van Nostrand Reinhold Company, New York, pp. 825-838.
Fine, A. S, Egnor, R. W. “Changes in cAMP phosphodiesterase activity during oral mucosal regeneration,” J Oral Pathol 15(3):138-42 (March 1986).
Finn, B. E., Evenas, J., Drakenberg, T., Waltho, J. P., Thulin, E. and Forsen, S. “Calcium-induced structural changes and domain autonomy in calmodulin,” Nat. Struct. Biol., 2:, 777-. (1995).
Flynn, G. L., “Mechanism of Percutaneous Absorption from Physiochemical Evidence,” Percutaneous Absorption: Mechanisms-Methodology-Drug Delivery (Bronaugh, R. L., Maibach, H., Editors, Marcel Dekker, New York) 27-51 (1989).
Gerendasy D. (1999) Homeostatic tuning of Ca2+ signal transduction by members of the calpacitin protein family,” J. Neurosci. Res., 58:, 107-.
Hahn, K., DeBiasio, R., Taylor, D. L. “Patterns of elevated free calcium and calmodulin activation in living cells,” Nature 22; 359(6397):736-8 (October 1992).
Haiech, J., Klee, C. B. & Demaille, J. G. “Effects on cations on affinity of calmodulin for calcium: ordered binding of calcium ions allows the specific activation of calmodulin stimulated enzymes,” Biochemistry 20:3890-3897 (1981).
Heggers, J. P., Elzaim, H., Garfield, R., Goodheart, R, Listengarten, D., Zhao, J. and Phillips, L. G. “Effect of the combination of Aloe vera, nitroglycerin, and L-NAME on wound healing in the rat excisional model,” J Altern Complement Med;3(2):149-53 (Summer 1997).
Henzl, M. R. “The Promise of Transdermal Drug Delivery—Not Only for Reproductive Steroids,” Science 269(5225):850-853.
Higbee, R. G., Hazlett, L. D. “Topical ocular anesthetics affect epithelial cytoskeletal proteins of wounded cornea,” J Ocul Pharmacol 5(3):241-53 (Fall 1989).
Junginger, et al., “Visualization of Drug Transport Across Human Skin and the Influence of Penetration Enhancers,” Drug Permeation Enhancement (Hsieh, D. S., Editors, Marcel Dekker, Inc. New York) 59-89 (1994).
Jurado L. A., Chockalingam, P. S. and Jarrett, H. W. (1999) “Apo-calmodulin,” Physiol. Rev., 79:, 661-.
Karande, P., Jain, A. & Mitragotri, S. “Discovery of transdermal penetration enhancers by high-throughput screening,” Nature Biotechnology 22:192-197 (2004).
Kawasaki, H. & Kretsinger, R. H. (1994) “Calcium-binding proteins 1: EF-hands,” Protein Profile 1(4):343-517 (1994).
Kost, et al., “Ultrasound Effect on Transdermal Drug Delivery,” (Ben Gurion University Dept. of Chem. Engineering, Beer Sheva Israel) (MIT, Dept. of Applied Biological Sciences, Cambridge, Mass.) CRS (August 1986).
Kost and Langer, “Ultrasound-Mediated Transdermal Drug Delivery,” Topical Drug Bioavailability Bioequivalence and Penetration (Maibach, H. I., Shah, V. P., Editors, Plenum Press, New York) 91-104 (1993).
Koyama, M., Spicer, S. S., Schulte, B. A. “Immunohistochemical Localization of Ca2+/Calmodulin-dependent Protein Kinase IV in Outer Hair Cells,” J. Histochem. Cytochem. 47:7-12 (1999).
Lansdown, A. B. “Calcium: a potential central regulator in wound healing in the skin,” Wound Repair Regen. 10(5):271-85 (September-October 2002).
Lansdown, A. B., Sampson, B. & Rowe, A. “Sequential changes in trace metal, metallothionein and calmodulin concentrations in healing skin wounds,” J Anat. 195(Pt 3):375-86 (October 1999).
Levinson, H., Moyer, K. E., Saggers, G. C. and Ehrlich, H. P. “Calmodulin-myosin light chain kinase inhibition changes fibroblast-populated collagen lattice contraction, cell migration, focal adhesion formation, and wound contraction,” Wound Repair Regen. 12(5):505-511 (October 2004).
Levy, et al., “Effect of Ultrasound on Transdermal Drug Delivery to Rats and Guinea Pigs,” J. Clin. Invest. K. 83:2074-2078 (1989).
Li, Y., Lin, J. L., Reiter, R. S., Daniels, K., Soll, D. R., Lin, J. J. “Caldesmon mutant defective in Ca(2+)-calmodulin binding interferes with assembly of stress fibers and affects cell morphology, growth and motility,” J Cell Sci. 15; 117(Pt 16):3593-604 (July 2004).
Lin, W. Q., Cormier, M., Samiee, A., Griffen, A., Johnson, B., Teng, C. L., Hardee, G. E. and Daddona, P. E. “Transdermal Delivery of Antisense Oligonucleotides with Microprojection Patch (Macroflux) Technology,” Pharm. Res. 18(12):1,789-1,793 (2001).
Machluf and Kost, “Ultrasonically enhanced transdermal drug delivery. Experimental approaches to elucidate the mechanism,” J. Biomater. Sci. Polymer Edn. 5:147-156 (1993).
Meyer, R B, Kreis, W, Eschbach, J, O'Mara, V, Rosen, S, Sibalis, D. “Successful transdermal administration of therapeutic doses of a polypeptide to normal human volunteers,” Clin Pharmacol Ther 44: 607-612 (1988).
Mitragotri, S., Blankschtein, D. and Langer, R. “Ultrasound-mediated Transdermal Protein Delivery,” Science, 269(5,225):850-853 (1995).
Mitragotri, S., Coleman, M., Kost, J. and Langer, R. “Analysis of Ultrasonically Extracted Interstitial Fluid as a Predictor of Blood Glucose Levels,” J. Appl. Physiol., 89:961-966 (2000).
Mitragotri, et al., “A Mechanism Study of Ultrasonic-Enhanced Transdermal Drug Delivery,” J. Pharm. Sci. 84:697-706 (1995).
Mizumoto, T. “[Effects of the calcium ion on the wound healing process]” [Article in Japanese] Hokkaido Igaku Zasshi. 62(2):332-45 (March 1987).
Morimoto, Y., et al., “Prediction of Skin Permeability of Drugs: Comparison of Human and Hairless Rat Skin,” J. Pharm. Pharmacol. 44:634-639 (1991).
Nojima, H. and Sokabe, H., “Structure of a gene for Rat Calmodulin,” J. Mol. Biol., 193:439-445 (1987) (GI 55860 & 1334203).
Olanoff and Gibson, “Method to Enhance Intranasal Peptide Delivery,” Controlled Release Technology Pharmaceutical Application (Lee, et al. Editors, American Chemical Society)301-309 (1987).
Osborne, D. W., Henke, J. J. “Skin Penetration Enhancers Cited In the Technical Literature,” Pharmaceutical Technology:58-80 (November 1997).
Persechini, A., Cronk, B. “The relationship between the free concentrations of Ca2+ and Ca2+-calmodulin in intact cells,” J Biol Chem 274(11):6827-6830 (Mar. 12, 1999).
Petrus E. J. et al., “Randomized, Double-Masked, Placebo-Controlled, Clinical Study of the Effectiveness of Zinc Acetate Lozenges on Common Cold Symptoms in Allergy-Tested Subjects” Current Therapeutic Research; 59(9):595-607 (1998).
Potts and Guy, “Predicting Skin Permeability,” Pharm. Res. 9:663-669 (1992).
Prausnitz, et al., “Electroporation of mammalian skin: A mechanism to enhance transdermal drug delivery,” Proc. Natl. Acad. Sci. USA 90:10504-10508 (1993).
Quillen, W. S., “Phonophoresis: A Review of the Literature and Technique,” Athl. Train. 15:109-110 (1980).
Rasmussen, C. and Means, A. R., “Calmodulin, cell growth and gene expression,” TINS, 12(11):433-438 (1989).
Richey, M. L., Richey, H. K., Fenske, N. A., “Aging-Related Skin Changes: Development and Clinical Meaning,” Geriatrics 43(4):49-61 (April 1988).
Rogers, M. S., Strehler, E. E. “Calmodulin” in Guidebook to the Calcium-Binding Proteins, M. R. Celio, T. Pauls, B. Schwaller, eds. Oxford University Press, Oxford. 34-40 (1996).
Scott, E. R., Phipps, B. J., Gyory, R. J. and Padmanabhan, R. V. “Electrotransport Systems for Transdermal Delivery: A Practical Implementation of Iontophoresis,” Handbook of Pharmaceutical Controlled Release Technology, D L Wise (ed.), New York: Marcel Dekker, pp. 617-659 (2000).
Shaw, J E, Chandrasekaran, S K, Michaels, A S, Taskovict, L “Controlled transdermal delivery, in-vitro and in-vivo,” Maibach, H. I. (ed.). Animal Models in Dermatology, Churchill-Livingstone, New York, 138-146 (1975).
Shen,Y., Lee, Y.-S., Soelaiman, S., Bergson, P., Lu, D., Chen, A., Beckingham, K., Grabarek, Z., Mrksich, M., and Tang, W.-J. “Physiological calcium concentrations regulate calmodulin binding and catalysis of adenylyl cyclase exotoxins,” EMBO J. 21(24):6721-6732 (Dec. 16 2002).
Skauen, et al., “Phonophoresis,” Int. J. Pharm. 20:235-245 (1984).
Soong, H. K, Cintron, C. “Different corneal epithelial healing mechanisms in rat and rabbit: role of actin and calmodulin,” Invest Ophthalmol Vis Sci. 26(6):838-48 (June 1985).
Tran, P. O., Hinman, L. E., Unger, G. M., Sammak, P. J. “A wound-induced [Ca2+] increase and its transcriptional activation of immediate early genes is important in the regulation of motility,” Exp Cell Res. 246(2):319-26 (Feb. 1 1999.)
Tyle and Agrawala, “Drug Delivery by Phonophoresis,” Pharm. Res. 6:355-361 (1989).
Veilalrd, et al., “Buccal Controlled Delivery of Peptides,” Proceed. Intern. Symp. Control. Rel. Bioact. Mater. (Controlled Release Society, Inc.) 14:6 (1987).
Walters, K. A., “Penetration Enhancers and Their Use in Transdermal Therapeutics Systems,” Transdermal Drug Delivery: Developmental Issues and Research Initiatives, 197-246 (Hadgraft J., Guy, R. H., Editors, Marcel Dekker, 1989).
Weiss, J. S., Ellis, C. N., Headington, J. T., Tincoff, T., Hamilton, T. A., Voorhees, J. J. “Topical tretinoin improves photoaged skin. A double-blind vehicle-controlled study,” JAMA. 259(4):527-532 (Jan. 22 1988).
Wester and Mailbach, “Animal Models for Percutaneous Absorption,” Topical Drug Bioavailability Bioequivalence and Penetration (Shah and Maibach, Editors, Plenum Press, New York) 333-349 (1993).
Williams, et al., “On the non-Gaussian distribution of human skin permeabilities,” Int. J. Pharm. 86:69-77 (1992).
Wilschut, et al., “Estimating Skin Permeation, The Validation of Five Mathematical Skin Permeation Models,” Chemosphere 30:1275-1296 (1995).
Wilson, Y., Goberdhan, N., Dawson, R. A., Smith. J., Freedlander, E. and Mac Neil, S. “Investigation of the presence and role of calmodulin and other mitogens in human bum blister fluid,” J. Bum Care Rehabil. 15(4):303-314 (1994).
Yang, S., Huang, X. Y. “Ca2+ influx through L-type Ca2+ channels controls the trailing tail contraction in growth factor-induced fibroblast cell migration,” J Biol Chem. 280(29):27130-7 (Jul. 22 2005.)

### U.S. Patent Documents:

| | | | |
|---|---|---|---|
| U. S. Patent #: | 4,296,130 | Herschler | October 1981 |
| U. S. Patent #: | 4,342,784 | Havemeyer at al. | August 1982 |
| U. S. Patent #: | 4,477,469 | Herschler | October 1984 |
| U. S. Patent #: | 4,704,280 | Bates | November 1987 |
| U. S. Patent #: | 4,772,591 | Meisner | September 1988 |
| U. S. Patent #: | 4,863,748 | Herschler | September 1989 |
| U. S. Patent #: | 4,933,184 | Tsuk | June 1990 |
| U. S. Patent #: | 4,938,969 | Schinitsky at al. | July 1990 |
| U. S. Patent #: | 4,973,605 | Herschler | November 1990 |
| U. S. Patent #: | 5,071,878 | Herschler | December 1991 |
| U. S. Patent #: | 5,196,417 | Dolling at al. | March 1993 |
| U. S. Patent #: | 5,229,130 | Sharma at al. | July 1993 |
| U. S. Patent #: | 5,238,933 | Catz at al. | August 1993 |
| U. S. Patent #: | 5,254,343 | Parah at al. | October 1993 |
| U. S. Patent #: | 5,281,722 | Blaschke at al. | January 1994 |
| U. S. Patent #: | 5,409,693 | Perricone | April 1995 |
| U. S. Patent #: | 5,449,688 | Wahl at al. | September 1995 |
| U. S. Patent #: | 5,468,476 | Ahluwalla at al. | November 1995 |
| U. S. Patent #: | 5,470,874 | Lerner | November 1995 |
| U. S. Patent #: | 5,482,965 | Rajadhyaksha | January 1996 |
| U. S. Patent #: | 5,569,670 | Weischer at al. | October 1996 |
| U. S. Patent #: | 5,667,791 | Hersh at al. | September 1997 |
| U. S. Patent #: | 5,691,380 | Mason at al. | November 1997 |
| U. S. Patent #: | 5,728,735 | Ulrich at al. | March 1998 |
| U. S. Patent #: | 5,747,536 | Cavazza | May 1998 |
| U. S. Patent #: | 5,780,693 | Bernhard at al. | July 1998 |
| U. S. Patent #: | 5,786,342 | Carpenter at al. | July 1998 |
| U. S. Patent #: | 5,789,396 | Blank at al. | August 1998 |
| U. S. Patent #: | 5,792,449 | Bryce-Smith | August 1998 |
| U. S. Patent #: | 5,804,594 | Murad | September 1998 |
| U. S. Patent #: | 5,807,957 | Samour at al. | September 1998 |
| U. S. Patent #: | 5,821,237 | Bissett at al. | October 1998 |
| U. S. Patent #: | 5,824,659 | Strickland at al. | October 1998 |
| U. S. Patent #: | 5,837,289 | Grasela at al. | November 1998 |
| U. S. Patent #: | 5,846,996 | Fallick | December 1998 |
| U. S. Patent #: | 5,869,539 | Garfield at al. | February 1999 |
| U. S. Patent #: | 5,876,736 | Cohen at al. | March 1999 |
| U. S. Patent #: | 5,883,128 | Yu at al. | March 1999 |
| U. S. Patent #: | 5,889,054 | Yu at al. | March 1999 |
| U. S. Patent #: | 5,922,331 | Mausner | July 1999 |
| U. S. Patent #: | 5,922,346 | Hersh | July 1999 |
| U. S. Patent #: | 5,925,348 | Riley at al. | July 1999 |
| U. S. Patent #: | 5,925,620 | Ohlenschlager at al. | July 1999 |
| U. S. Patent #: | 5,939,394 | Fleming at al. | August 1999 |
| U. S. Patent #: | 5,945,447 | Fallick | August 1999 |
| U. S. Patent #: | 6,573,299 | Petrus | June 2003 |
| U. S. Patent #: | 6,743,449 | Pinnell et al. | June 2004 |
| U. S. Patent #: | 6,989,368 | McMichael et al. | January 2006 |
| U. S. Patent #: | 7,060,303 | Jones | June 2006 |
| U. S. Patent #: | 7,148,194 | Malik et al. | December 2006 |

I claim:

1. Compositions for topical application to skin and/or scalp, comprising, in a physiologically and dermatologically acceptable medium, calmodulin (CaM) protein or its derivatives with or without other bio-affecting agents or permeation enhancers.

2. The composition according to claim 1, further comprising at least one active agent chosen from desquamating agents; moisturizers; depigmenting and propigmenting agents; anti-glycation agents; nitric oxide synthetase inhibitors (NO-SI); agents for stimulating the synthesis of dermal or epidermal macromolecules and/or for preventing their degradation; agents for stimulating fibroblast and/or keratinocyte proliferation or for stimulating keratinocyte differentiation; dermo-relaxants; tightening agents; antipollution agents and free-radical scavengers; agents that act on the capillary circulation; and agents that act on the energy metabolism of cells.

3. The composition according to claim 1 and/or claim 2, wherein the composition is in a form chosen from an optionally gelled solution, a cream, a serum, an ointment, an oil, a foundation, a tonic, a depilatory, a patch, through spraying, a lotion dispersion, optionally a two-phase lotion, an oil-in-water (O/W) emulsion, a water-in-oil (W/O) emulsion, a triple emulsion chosen from water-in-oil-in-water (W/O/W) or oil-in-water (O/W/O) emulsions, and ionic and nonionic vesicular dispersions.

4. The composition according to claim 1 and/or claim 2 and/or claim 3, which contains urea for softening and moisturizing dry, rough, cracked or calloused skin.

5. The composition according to claim 1 and/or claim 2 and/or claim 3, further comprising at least one photoprotective agent chosen from UVA-active and UVB-active organic and mineral photoprotective agents for the prevention of wrinkles and fine lines.

6. The composition according to claim 5, wherein at least one photoprotective agent is present in an amount ranging from 0.1% to 20% by weight of the total weight of the composition.

7. The composition according to claim 1, and/or claim 2, and/or claim 3, and/or claim 4, and/or claim 5, and/or claim 6 further comprising at least one component selected from the group consisting of hydrophilic and lipophilic gelling agents, hydrophilic and lipophilic active agents, emulsifiers, fillers, solvents, preservatives, antioxidants, odor absorbers, colorants, fragrances and combinations thereof.

8. The composition according to claim 1, and/or claim 2, and/or claim 3, and/or claim 4, and/or claim 5, and/or claim 6, and/or claim 7 comprising at least one penetration enhancer and/or at least one alpha hydroxy acid (AHA) and/or beta hydroxy acid (BHA), in a therapeutically acceptable vehicle, facilitating the delivery of bio-affecting agents through the protective outer layer of the skin into the underlying tissues and into the vascular network.

9. A method of making a cosmetic composition for topical application to skin comprising, in the composition, CaM with/or without comprising constituents as described in claim 2 and/or claim 3 and/or claim 4 and/or claim 5 and/or claim 6 and/or claim 7 and/or claim 8.

10. A method of making a cosmetic composition for topical application to skin according to claim 8, wherein said penetration enhancer are chemicals such as, but not limited to, those selected chemicals such as: alcohols, polyols, sulfoxides, esters, ketones, amides, oleates, surfactants, alkanoic acids, lactam compounds, alkanols, dialkylamino acetates, and mixtures thereof.

11. The method of claim 10, wherein topically applying to skin comprises a transdermal patch application.

12. A method of applying the cosmetic composition as described in claim 1 and/or claim 2 and/or claim 3 and/or claim 4 and/or claim 5 and/or claim 6 and/or claim 7 and/or claim 8 through non-chemically mediated transdermal delivery processes such as, but not exclusive to, the use of sonophoresis and/or electric potential and/or other transdermal effectors for larger peptides and proteins.

13. A method of making a cosmetic composition for topical application to skin comprising, in the composition, CaM for preventing or treating wrinkles and fine lines.

14. The method according to claim 13 wherein the prevention or treatment of wrinkles and fine lines comprises smoothing out the wrinkles and fine lines.

15. The method according to claim 13 and/or claim 14, wherein the wrinkles are expression wrinkles.

16. A cosmetic process for preventing wrinkles and fine lines on skin, comprising topically applying a composition, as described in claim 1, and/or claim 2, and/or claim 3, and/or claim 4, and/or claim 5, and/or claim 6, and/or claim 7, and/or claim 8, to the skin, wherein the composition comprises, in a physiologically acceptable medium, CaM.

17. The cosmetic process according to claim 16, wherein the wrinkles are expression wrinkles.

18. The process according to claim 17, wherein said composition is applied to the areas of the face or forehead marked with expression wrinkles and fine lines and/or to individuals with expression wrinkles and fine lines.

19. The process according to claim 18, wherein said composition is applied to the wrinkles and fine lines lying radially around the mouth and/or the eyes and/or horizontally on the forehead and/or in the space between the eyebrows and/or around the jowl.

20. A cosmetic process facilitating minor wound healing comprising topically applying a composition, as described in claim 1, and/or claim 2, and/or claim 3, and/or claim 4, and/or claim 5, and/or claim 6, and/or claim 7, and/or claim 8, to the skin, wherein the composition comprises, in a physiologically acceptable medium, CaM.

21. A cosmetic process facilitating burn healing comprising topically applying a composition, as described in claim 1, and/or claim 2, and/or claim 3, and/or claim 4, and/or claim 5, and/or claim 6, and/or claim 7, and/or claim 8, to the skin, wherein the composition comprises, in a physiologically acceptable medium, CaM.

22. A cosmetic process hair growth enhancement comprising topically applying a composition, as described in claim 1, and/or claim 2, and/or claim 3, and/or claim 4, and/or claim 5, and/or claim 6, and/or claim 7, and/or claim 8, to the scalp in particular, or other areas where hair growth is desired, wherein the composition comprises, in a physiologically acceptable medium, CaM.
